(19) United States
(12) Patent Application Publication
(10) Pub. No.: US 2025/0186071 A1
(43) Pub. Date: Jun. 12, 2025

(54) THROMBECTOMY CATHETER SYSTEM AND METHODS OF USE

(71) Applicant: Walk Vascular, LLC, Irvine, CA (US)

(72) Inventors: Nianjiong Bei, Palo Alto, CA (US); Seung Hae Clare Chung, Los Altos, CA (US); Jared Ryan G. Lumauig, San Jose, CA (US); Dustin M. Cossio, Brea, CA (US); Nora V. Legarda, San Jose, CA (US); Ted Su, Sunnyvale, CA (US); Matthew J. Gillick, Murrieta, CA (US)

(21) Appl. No.: 18/971,460

(22) Filed: Dec. 6, 2024

Related U.S. Application Data
(60) Provisional application No. 63/607,448, filed on Dec. 7, 2023.

(51) Int. Cl. A61B 17/22; A61B 17/3203

(52) U.S. Cl. CPC A61B 17/22; A61B 17/32037; A61B 2017/22079

(57) ABSTRACT

A thrombectomy system comprising an aspiration catheter having an aspiration lumen and a supply lumen, the aspiration catheter having a proximal end and a distal end, and a drive unit fluidly connected to the aspiration catheter and configured to provide pressurized fluid and vacuum through the aspiration catheter, wherein, the aspiration catheter comprises a distal opening and wherein the supply lumen has at least one orifice, wherein the at least one distal orifice of the supply lumen is proximal of the distal opening of the aspiration catheter, so as to be proximally disposed relative to the distal opening between about 0.010 inches and about 50 inches.

### CROSS-REFERENCE TO RELATED APPLICATIONS

This application claims the benefit of and priority to U.S. Provisional Patent Application Ser. No. 63/607,448 filed Dec. 7, 2023, and entitled “THROMBECTOMY CATHETER SYSTEM AND METHODS OF USE,” which application is expressly incorporated herein by reference in its entirety.

### BACKGROUND

### 1. The Field of the Invention

The present disclosure relates to removal of clots in blood vessels, which employ a combination of suction through a catheter and an internal high-pressure saline jet within the catheter to macerate clot tissue during aspiration.

### 2. The Relevant Technology

Several devices and systems already exist to aid in the removal of thrombotic material. These include simple aspiration tube type devices using vacuum syringes to extract thrombus into the syringe, simple flush-and-aspirate devices, more complex devices with rotating components that pull in, macerate and transport thrombotic material away from the distal tip using a mechanical auger, and systems that use very high pressure to macerate the thrombus and create a venturi effect to flush the macerated material away.

All of the devices described above have limitations as a result of individual design characteristics. For example, simple aspiration catheters offer ease of use and rapid deployment but may become blocked or otherwise inoperable when faced with older, more organized thrombotic material. Such devices must be removed and cleared outside the body and then re-inserted into the vasculature, which lengthens the time needed for the procedure and increases the opportunity to kink the catheter shaft.

### BRIEF SUMMARY OF THE INVENTION

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

An embodiment of the present disclosure relates to a thrombectomy system, comprising an aspiration catheter having an aspiration lumen and a supply lumen, the aspiration catheter having a proximal end and a distal end; and a drive unit fluidly connected to the aspiration catheter and configured to provide pressurized fluid and vacuum through the aspiration catheter, wherein, the aspiration catheter comprises a distal opening and wherein the supply lumen has at least one orifice, wherein the distal orifice of the supply lumen is proximal of the distal opening of the aspiration catheter, so as to be proximally disposed relative to the distal opening, between about 0.035 inches and about 0.060 inches. While the supply lumen carrying the pressurized fluid may principally be described as running within the aspiration catheter, this is not necessarily required (e.g., the supply lumen could run outside, e.g., parallel to the aspiration lumen for part or most of its length).

Another embodiment of the present disclosure relates to a method of removing a thrombus, comprising the steps of providing a thrombectomy system, comprising an aspiration catheter having an aspiration lumen and a supply lumen, the aspiration catheter having a proximal end and a distal end; and a drive unit fluidly connected to the aspiration catheter and configured to provide pressurized fluid and vacuum through the aspiration catheter, wherein, the aspiration catheter comprises a distal opening and wherein the supply lumen has at least one orifice, wherein the distal orifice of the supply lumen is proximal of the distal opening of the aspiration catheter, so as to be proximally disposed relative to the distal opening, between about 0.035 inches and about 0.060 inches. The method further includes inserting the aspiration catheter percutaneously into a body vessel lumen; advancing the aspiration catheter to a region of interest; and operating the drive unit to provide pressurized fluid and aspiration through the aspiration catheter to draw the thrombus into the aspiration lumen.

An embodiment of the present disclosure relates to a thrombectomy system, comprising an aspiration catheter having an aspiration lumen and a supply lumen, the aspiration catheter having a proximal end and a distal end; and a drive unit fluidly connected to the aspiration catheter and configured to provide pressurized fluid and vacuum through the aspiration catheter, wherein, the aspiration catheter comprises a distal opening and wherein the supply lumen has at least one orifice, wherein the distal orifice of the supply lumen is proximal of the distal opening of the aspiration catheter, so as to be proximally disposed relative to the distal opening, between about 0.010 inches and about 50 inches.

Another embodiment of the present disclosure relates to a method of removing a thrombus, comprising the steps of providing a thrombectomy system, comprising an aspiration catheter having an aspiration lumen and a supply lumen, the aspiration catheter having a proximal end and a distal end; and a drive unit fluidly connected to the aspiration catheter and configured to provide pressurized fluid and vacuum through the aspiration catheter, wherein the supply lumen has at least one orifice, wherein the distal orifice of the supply lumen is proximal of the distal opening of the aspiration catheter, so as to be proximally disposed relative to the distal opening, between about 0.010 inches and about 50 inches. The method further includes inserting the aspiration catheter percutaneously into a body vessel lumen; advancing the aspiration catheter to a region of interest; and operating the drive unit to provide pressurized fluid and aspiration through the aspiration catheter to draw the thrombus into the aspiration lumen.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

A description of various aspects and features of the invention will be rendered by reference to various representative embodiments thereof illustrated in the appended drawings. It is appreciated that these drawings depict only typical embodiments of the invention and are therefore not to be considered limiting of its scope.

FIG. 1 is a plan view of exemplary disposable components of a system for aspirating thrombus according to an implementation of the present disclosure.

FIG. 2 is a cross-sectional view of an exemplary distal end of the aspiration catheter of the system for aspirating thrombus of FIG. 1 according to an implementation of the present disclosure.

FIG. 3 is a detail view of an exemplary y-connector of the aspiration catheter of the system for aspirating thrombus of FIG. 1 according to an implementation of the present disclosure.

FIG. 4 is a perspective view of an exemplary system for aspirating thrombus using system components such as those of FIG. 1 according to an implementation of the present disclosure.

FIG. 5 is an exploded view of a portion of the system of FIG. 4 according to an implementation of the present disclosure.

FIG. 6 is a schematic representation of the aspiration system according to an implementation of the present disclosure.

FIG. 7 is a perspective view of an exemplary aspiration catheter according to an implementation of the present disclosure.

FIG. 8 is a cross-sectional view of an exemplary aspiration catheter according to an implementation of the present disclosure.

FIG. 9 schematically illustrates the exemplary aspiration catheter within a body lumen aspirating thrombus according to an implementation of the present disclosure.

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to a thrombectomy system configured to aspirate and macerate thrombus. For instance, the thrombectomy system can include an aspiration catheter having an aspiration lumen and a supply lumen, the aspiration catheter having a proximal end and a distal end; and a drive unit fluidly connected to the aspiration catheter and configured to provide pressurized fluid and vacuum through the aspiration catheter, wherein the supply lumen has at least one orifice, wherein the distal orifice of the supply lumen is proximal of the distal opening of the aspiration catheter, so as to be proximally disposed relative to the distal opening, between about 0.010 inches and about 50 inches. In an embodiment, the supply lumen carrying the pressurized fluid runs within the aspiration catheter, although this is not necessarily required (e.g., the supply lumen could run outside, e.g., parallel to the aspiration lumen for part or most of its length).

While the present disclosure will describe various particular implementations of a thrombectomy system configured to aspirate and macerate thrombus, it should be understood that the devices, systems, and method described herein may be applicable in other environments, and to other uses. Additionally, elements described in relation to any embodiment depicted and/or described herein may be combinable with elements described in relation to any other embodiment depicted and/or described herein.

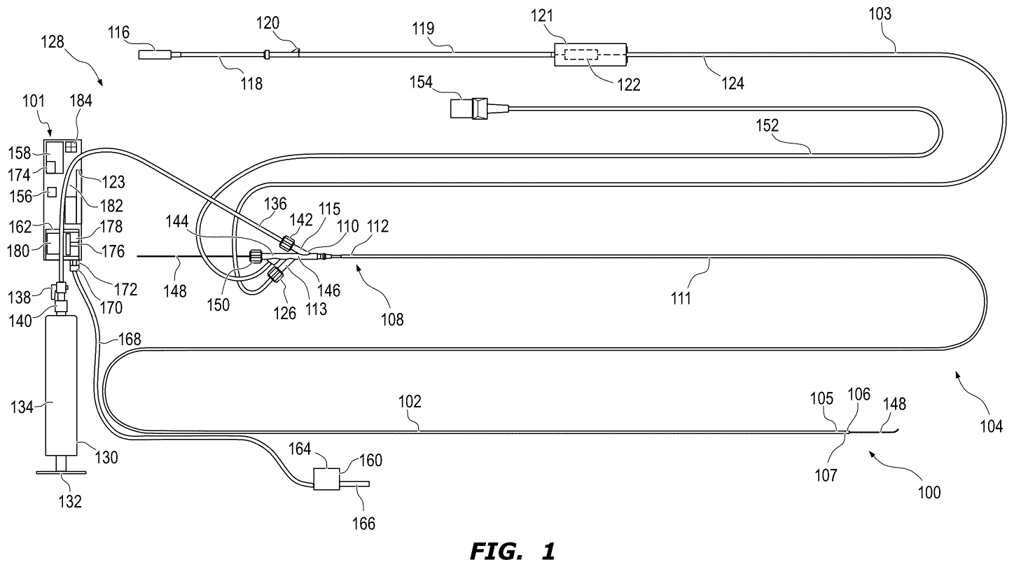

A system 100 for aspirating thrombus is illustrated in FIGS. 1-5, The system 100 for aspirating thrombus includes a pump 101, an aspiration catheter 102, and a tubing set 103. The aspiration catheter 102 and the tubing set 103 represent disposable components 104, and the pump 101, and the pump's associated pump base, is a reusable component. It is not necessary to sterilize the pump 101 as it may be kept in a non-sterile field or area during use. The aspiration catheter 102 and the tubing set 103 may each be supplied sterile, after sterilization by ethylene oxide gas, electron beam, gamma, or other sterilization methods. The aspiration catheter 102 may be packaged and supplied separately from the tubing set 103, or the aspiration catheter 102 and the tubing set 103 may be packaged together and supplied together. Alternatively, the aspiration catheter 102 and tubing set 103 may be packaged separately, but supplied together (i.e., bundled).

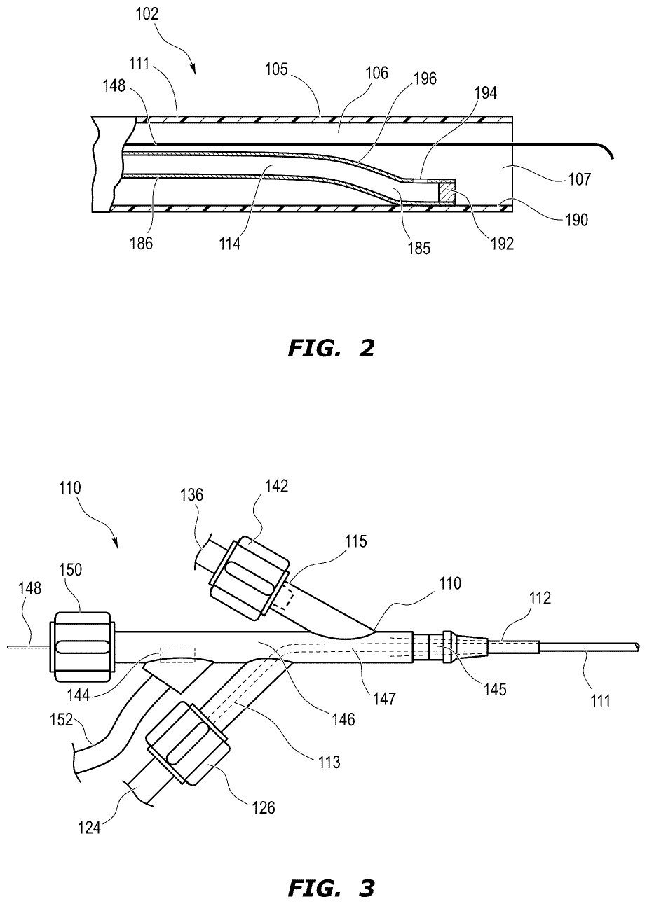

The aspiration catheter 102 has a distal end 105 and includes an over-the-wire guidewire lumen/aspiration lumen 106 extending between an open distal end 107, and a proximal end 108 comprising or connected to a y-connector 110. The catheter shaft 111 of the aspiration catheter 102 is connected to the y-connector 110 via a protective strain relief 112. In other embodiments, the catheter shaft 111 may be attached to the y-connector 110 with a luer fitting. The y-connector 110 includes a first female luer 113 which communicates with a catheter supply lumen 114 (FIG. 2), and a second female luer 115 which communicates with the guidewire lumen/aspiration lumen 106.

A spike 116 for coupling to a fluid source (not shown) (e.g., saline bag, saline bottle) allows fluid to enter through an extension tubing 118 and flow into a supply tube 119. An optional injection port 120 allows injection of materials or removal of air. A cassette 121 having a moveable piston 122 is used in conjunction with a mechanical actuator 123 of the pump 101. Fluid is pumped into an injection tube 124 from action of the cassette 121 as applied by the actuator 123 of the pump 101. A male luer 126, hydraulically communicating with the catheter supply lumen 114, via the injection tube 124, is configured to attach to the female luer 113 of the y-connector 110.

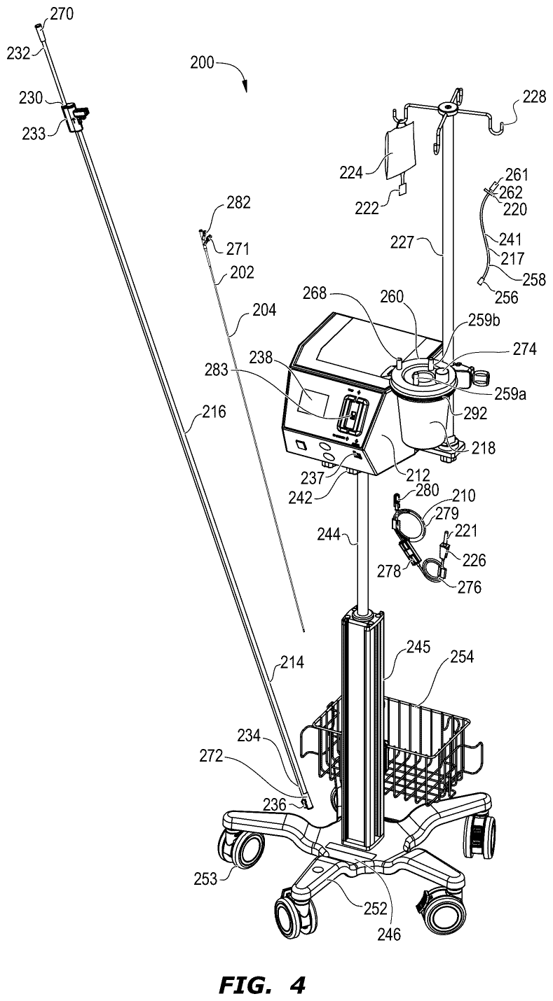

Accessories 128 are illustrated that are intended for applying a vacuum source, such as a syringe 130 having a plunger 132 and a barrel 134, to the aspiration lumen 106 of the aspiration catheter 102. The syringe 130 is attached to a vacuum line 136 via the luer 140 of the syringe 130. A stopcock 138 may be used on the luer 140 to maintain the vacuum, or alternatively, the plunger 132 may be a locking variety of plunger that is configured to be locked in the retracted (vacuum) position. A male luer 142 at the end of the vacuum line 136 may be detachably secured to the female luer 115 of the y-connector 110 of the aspiration catheter 102. As shown in more detail in FIG. 3, a pressure sensor or transducer 144 is secured inside an internal cavity 146 of the y-connector 110 proximal to the female luer 113 and the female luer 115. A valve 150, for example a Touhy-Borst, at the proximal end of the y-connector 110 allows hemostasis of the guidewire lumen/aspiration lumen 106 around a guidewire 148. In other embodiments, the valve 150 may comprise a longitudinally spring-loaded seal. The guidewire 148 may be inserted entirely through the guidewire lumen/aspiration lumen 106. Signals output from the pressure sensor 144 are carried through a cable 152 to a connector. The connector is plugged into a socket 156 of the pump 101. Pressure related signals may be processed by a circuit board 158 of the pump 101. The pressure sensor or transducer 144 may be powered from the pump 101, via the cable 152. The accessories 128 may also be supplied sterile to the user.

A foot pedal 160 is configured to operate a pinch valve 162 for occluding or opening the vacuum line 136. The foot pedal 160 comprises a base 164 and a pedal 166 and is configured to be placed in a non-sterile area, such as on the floor, under the procedure table/bed. The user steps on the pedal 166 causing a signal to be sent along a cable 168 which is connected via a plug 170 to an input jack 172 in the pump 101. The vacuum line 136 extends through a portion of the pump 101. The circuit board 158 of the pump may include a controller 174 configured to receive one or more signals indicating on or off from the foot pedal 160. The controller 174 of the circuit board 158, with associated processors, hardware, firmware, software, etc., may be configured to cause an actuator 176 carried by the pump 101 to move longitudinally to compress and occlude the vacuum line 136 between an actuator head 178 attached to the actuator 176 and an anvil 180, also carried by the pump 101. By stepping on the pedal 166, the user is able to thus occlude the vacuum line 136, stopping the application of a negative pressure. In some embodiments, as the pedal 166 of the foot pedal 160 is depressed, the controller may be configured to open the pinch valve 162.

The pressure sensor or transducer 144 thus senses a negative pressure and sends a signal, causing the controller to start the motor of the pump 101. As the effect via the electronics is substantially immediate, the motor starts pumping almost immediately after the pedal 166 is depressed. As the pedal 166 of the foot pedal 160 is released, the controller 174 then causes the pinch valve 162 to close. The pressure sensor or transducer 144 thus senses that no negative pressure is present and the controller 174 causes the motor of the pump 101 to shut off. Again, the effect via the electronics is substantially immediate, and thus the motor stops pumping almost immediately after the pedal 166 is depressed. During sterile procedures, the main interventionalist is usually “scrubbed” such that the hands only touch items in the sterile field. However, the feet/shoes/shoe covers are not in the sterile field. Thus again, a single user may operate a switch (via the pedal 166) while also manipulating the aspiration catheter 102 and guidewire 148. However, this time, it is the sterile field hands and non-sterile field feet that are used. Alternatively, the foot pedal 160 may comprise two pedals, one for occlude and one for open. In an alternative foot pedal embodiment, the pedal 166 may operate a pneumatic line to cause a pressure activated valve or a cuff to occlude and open the vacuum line 136, for example, by forcing the actuator head 178 to move. In another alternative embodiment, the pedal 166 may turn, slide, or otherwise move a mechanical element, such as a flexible pull cable or push rod that is coupled to the actuator 176, to move the actuator head 178. The cable 168 may be supplied sterile and connected to the base 164 prior to a procedure. The occlusion and opening of the vacuum line 136 thus acts as an on and off switch for the pump 101 (via the pressure sensor 144). The on/off function may thus be performed by a user whose hands can focus on manipulating sterile catheters, guidewires, and accessories, and whose foot can turn the pump on and off in a non-sterile environment. This allows a single user to control the entire operation or the majority of operation of the system 100 for aspirating thrombus. This can be an advantage both in terms of a rapid, synchronized procedure, but is also helpful in laboratories where additional assistants are not available. The actuator 176 and anvil 180 may be controlled to compress the vacuum line 136 with a particular force, and the actuator 176 may be controlled to move at a particular speed, either when compressing or when removing compression. Speed and force control allows appropriate response time, but may also be able to add durability to the vacuum line 136, for example, by not over-compressing. The foot pedal 160 may communicate with the pinch valve 162 via a wired connection through the pump 101 or may communicate with the pinch valve 162 wirelessly. Additionally, or alternatively, the pump may be controlled by buttons 184.

It should be noted that in certain embodiments, the pinch valve 162 and the foot pedal 160 may be incorporated for on/off operation of the pinch valve 162 on the vacuum line 136, without utilizing the pressure sensor 144. In fact, in some embodiments, the pressure sensor 144 may even be absent from the system 100 for aspirating thrombus, the foot pedal 160 being used as a predominant control means.

Turning to FIG. 2, a supply tube 186, which contains the catheter supply lumen 114, freely and coaxially extends within the over-the-wire guidewire lumen/aspiration lumen 106. At least a distal end 185 of the supply tube 186 is secured to an interior wall 190 of the guidewire lumen/aspiration lumen 106 of the catheter shaft 111 by adhesive, epoxy, hot melt, thermal bonding, or other securement modalities. A plug 192 is secured within the catheter supply lumen 114 at the distal end 185 of the supply tube 186. The plug 192 blocks the exit of pressurized fluid, and thus the pressurized fluid is forced to exit through an orifice 194 in the wall 196 of the supply tube 186. The free, coaxial relationship between the supply tube 186 and the catheter shaft 111 along their respective lengths, allows for improved flexibility. In some embodiments, in which a stiffer proximal end of the aspiration catheter 102 is desired (e.g., for pushability or even torqueability), the supply tube 186 may be secured to the interior wall 190 of the guidewire lumen/aspiration lumen 106 of the catheter shaft 111 along a proximal portion of the aspiration catheter 102, but not along a distal portion. This may be appropriate if, for example, the proximal portion of the aspiration catheter 102 is not required to track through tortuous vasculature, but the distal portion is required to track through tortuous vasculature. The free, substantially unconnected, coaxial relationship between the supply tube 186 and the catheter shaft 111 along their respective lengths, may also be utilized to optimize flow through the guidewire lumen/aspiration lumen 106, as the supply tube 186 is capable of moving out of the way due to the forces of flow (e.g., of thrombus/saline) over its external surface, such that the remaining inner luminal space of the guidewire lumen/aspiration lumen 106 self-optimizes, moving toward the lowest energy condition (least fluid resistance) or toward the largest cross-sectional space condition (e.g., for accommodating and passing pieces of thrombus).

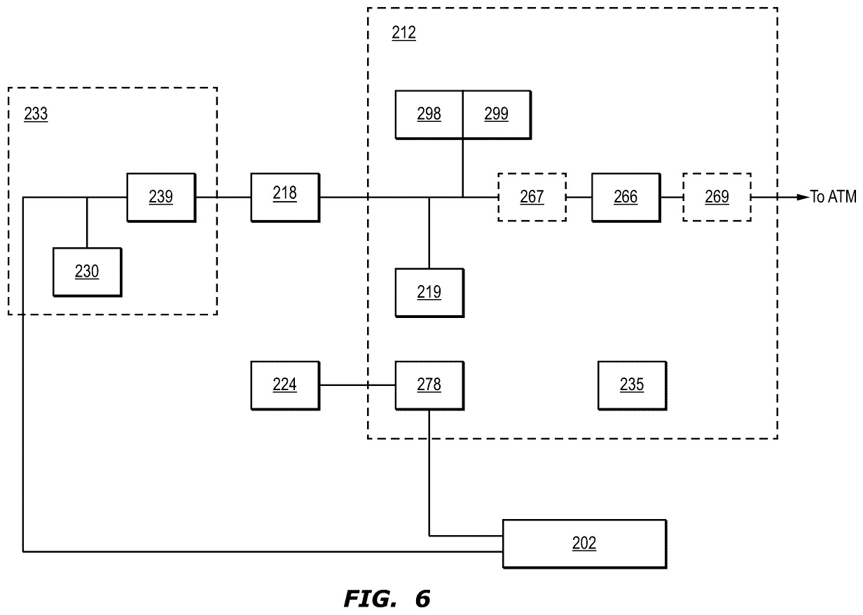

A system 200 for aspirating thrombus is illustrated in FIGS. 4-6. The system 200 for aspirating thrombus is similar to the system 100 and so the disclosure related to the system 100 is also applicable to the description of system 200. An aspiration catheter 202 is similar to the aspiration catheter 102 of FIGS. 1-3 and as such the description related to the aspiration catheter 102 of FIGS. 1-3 is also applicable to the description of the aspiration catheter 202. The system 200 of FIG. 6 is schematically illustrated with functional blocks associated with the functions of structures described herein.

The aspiration catheter 202 is configured for aspirating thrombus from peripheral vessels, but may also be configured with a size for treating coronary, cerebral, pulmonary or other arteries, or veins. The aspiration catheter 202 and system 200 may be used in interventional procedures, but may also be used in surgical procedures. The aspiration catheter 202 and system 200 may be used in vascular procedures, or non-vascular procedures (other body lumens, ducts, or cavities). The catheter 202 comprises an elongate shaft 204 configured for placement within a blood vessel of a subject; a catheter supply lumen 114 (FIGS. 2-3) and a guidewire/aspiration lumen 106, each extending along the shaft, the supply lumen 114 having a proximal end 147 and a distal end 185, and the aspiration lumen 106 having a proximal end 145 (FIG. 3) and an open distal end 107 (FIG. 2); and an orifice or opening 194 at or near the distal end 185 of the supply lumen 114, the opening configured to allow the injection of pressurized fluid into the aspiration lumen 106 at or near the distal end 107 of the aspiration lumen 106 when the pressurized fluid is pumped through the supply lumen 114 (FIG. 2). In some embodiments, the orifice or opening 194 may be located proximal to the distal end 185 of the supply lumen 114. In some embodiments, the distal end 185 of the supply lumen 114 may comprise a plug 192.

The orifice 194 may have a diameter between about 0.001 inches and about 0.006 inches, or between about 0.002 inches and about 0.004 inches, or about 0.0031 inches. As another example, the orifice may comprise a generally rectangular hole with each side thereof having a length between about 0.0008 inches and about 0.008 inches. The diameter of the supply lumen 114 may be between about 0.3048 mm (0.012 inches) and about 0.4826 mm (0.019 inches), or between about 0.3556 mm (0.014 inches and about 0.4318 mm (0.017 inches) or about 0.3937 mm (0.0155 inches). The orifice 194 is set proximally of the open distal end 107 by a set amount. For example, orifice 194 can be set proximally of the open distal end 107 by about 0.040 inches, and in one configuration by 0.051±0.003 inches or by another desired amount. For example, orifice 194 can be set proximally of the open distal end 107 by approximately 0.035 inches, 0.036 inches, 0.037 inches, 0.038 inches, 0.039 inches, 0.040 inches, 0.041 inches, 0.042 inches, 0.043 inches, 0.044 inches, 0.045 inches, 0.046 inches, 0.047 inches, 0.048 inches, 0.049 inches, 0.050 inches, 0.051 inches, 0.052 inches, 0.053 inches, 0.054 inches, 0.055 inches, 0.056 inches, 0.057 inches, 0.058 inches, 0.059 inches, 0.060 inches, or a range defined by any two of the foregoing. In still other configurations, the open distal end 107 can be set proximally of the open distal end 107 by about 0.01 inches to about 50 inches, between about 0.010 inches and about 40 inches, or between about 0.010 inches and about 2 inches.

In still another configuration a diameter of the aspiration lumen 106 is about 0.075 inches to about 0.177 inches, the orifice 194 (and so a distal end of the supply tube 186) can be set proximally of open distal end 107 about 12 inches. In still another configuration a diameter of the aspiration lumen 106 is about 0.075 inches, the orifice 194 (and so a distal end of the supply tube 186) can be set proximally of open distal end 107 about 0.035 inches to about 0.060 inches. In still another configuration, the orifice 194 (and so a distal end of the supply tube 186) can be set proximally of open distal end 107 about 0.010 inches to about 50 inches where the catheter 204 has a catheter size ranging from about 3 Fr to about 50 Fr. In still another configuration, the orifice 194 (and so a distal end of the supply tube 186) can be set proximally of open distal end 107 about 0.010 inches to about 0.200 inches, from about 0.010 inches to about 2 inches, 0.010 inches to about 40 inches, 0.010 inches to about 50 inches, or a range defined by any two of the foregoing. In still other configurations, the orifice 194 (and so a distal end of the supply tube 186) can be set proximally of open distal end 107 based upon Table 1:

TABLE 1

| Catheter size | Distal Orifice Proximal Offset (range in inches) |
|---|---|
| 3 Fr | 0.01 to 0.200 |
| 4 Fr | |
| 5 Fr | |
| 6 Fr | |
| 7 Fr | |
| 8 Fr | |
| 9 Fr | 0.01 to 2 |
| 10 Fr | |
| 11 Fr | |
| 12 Fr | 0.01 to 40 |
| 13 Fr | |
| 14 Fr | |
| 15 Fr | |
| 16 Fr | |
| 17 Fr | 0.01 to 50 |
| 18 Fr | |
| 19 Fr | |
| 20 Fr | |
| 21 Fr | |
| 22 Fr | |
| 23 Fr | |
| 24 Fr | |
| 25 Fr | |
| 26 Fr | |

In another configuration, the orifice 194 (and so a distal end of the supply tube 186) can be set proximally of open distal end 107 about 0.010 inches to about 0.200 inches for a catheter having a catheter size ranging from about 3 Fr to about 8 Fr. In another configuration, the orifice 194 (and so a distal end of the supply tube 186) can be set proximally of open distal end 107 about 0.010 inches to about 0.200 inches for a catheter having a catheter size ranging from about 9 Fr to about 11 Fr. In another configuration, the orifice 194 (and so a distal end of the supply tube 186) can be set proximally of open distal end 107 about 0.010 inches to about 40 inches for a catheter having a catheter size ranging from about 12 Fr to about 16 Fr. In another configuration, the orifice 194 (and so a distal end of the supply tube 186) can be set proximally of open distal end 107 about 0.010 inches to about 50 inches for a catheter having a catheter size ranging from about 17 Fr to about 26 Fr. In other configurations, the ranges can include any combination of a range defined by any two of the foregoing proximal location of the orifice 194 with a range defined by any two of the foregoing catheter sizes.

A pump set 210 (e.g., tubing set) is configured to hydraulically couple the supply lumen 114 to a pump within a saline drive unit (SDU) 212, for injecting pressurized fluid (e.g., saline, heparinized saline) through the supply lumen 114. Suction tubing 214, comprising sterile suction tubing 216 and non-sterile suction tubing 217, is configured to hydraulically couple a vacuum canister 218 to the aspiration lumen 106. A filter 220 may be carried in-line on the suction tubing 214, for example, connected between the sterile suction tubing 216 and the non-sterile suction tubing 217, or on the non-sterile suction tubing 217. The filter 220 is configured to capture large elements such as large pieces of thrombus or emboli.

The pump set 210 includes a saline spike 221 for connection to a port 222 of a saline bag 224, and an inline drip chamber 226 for visually assessing the movement of saline, as well as keeping air out of the fluid being injected. The saline bag 224 may be hung on an IV pole 227 on one or more hooks 228. A pressure sensor 230 such as a vacuum sensor may be used within any lumen of the pump set 210, the suction tubing 214, the supply lumen 114 or aspiration lumen 106 of the catheter 202, or any other component which may see fluid flow. The pressure sensor 230 is shown in FIG. 4 within a lumen at a junction between a first aspiration tube 232 and a control 233; the control 233 including an operable valve 239 through which fluid flows to the supply lumen 114. A cable 234 carries signals output from the pressure sensor 230 to a controller 235 in the SDU 212. A connector 236, electrically connected to the cable 234, is configured to be detachably coupled to a mating receptacle 237 (e.g., input jack) in the SDU 212. The SDU 212 also may have a display 238, including an LCD screen or alternative screen or monitor, in order to visually monitor parameters and status of a procedure. In alternative embodiments, the pressure sensor 230 may be replaced by another type of sensor that is configured to characterize fluid flow. In some embodiments, the sensor is a flow sensor, such as a Doppler flow velocity sensor.

The SDU 212 is held on a mount 240 by four locking knobs 242. The mount 240 is secured to a telescoping rod 244 that is adjustable from a cart base 245 via a cart height adjustment knob or other element 246. The mount 240 and a handle 247 are secured to the rod 244 via an inner post 248 that is insertable and securable within an inner cavity in the rod 244. The IV pole 227 secures to the mount 240 via a connector 250. The base 245 may include legs 252 having wheels 253 (e.g., three or more wheels or four or more wheels) and may be movable via the handle 247. The system 200 may also carry a basket 254 for placement of components, products, documentation, or other items.

In use, a user connects a first connector 256 at a first end 258 of the non-sterile suction tubing 217 to a second port 259b on the lid 260 of the canister 218, and connects a second connector 261 at a second end 262 of the non-sterile suction tubing 217 to a vacuum pump input 264 in the SDU 212. A vacuum pump 266 may be carried within the SDU 212 in order to maintain a vacuum/negative pressure within the canister 218.

Alternatively, the vacuum inside the canister 218 may be maintained manually, without a vacuum pump, by evacuating the canister 218 via one or more additional ports 268. The vacuum pump 266 communicates with atmosphere through a manifold and/or filter 269.

In another configuration, a vacuum regulator 267 is disposed between the vacuum pump 266 and the canister 218, optionally in-line between the canister 218 and the SDU 212, to adjust or reduce the vacuum level generated by the vacuum pump 266. For instance, while the vacuum pump 266 can generate in excess of −29.5 inHg (expressed as gauge pressure readings relative to atmospheric pressure (not absolute values)) vacuum at sea level and −24.5 inHg (expressed as gauge pressure readings relative to atmospheric pressure (not absolute values)) at about 5280 feet elevation, for certain procedures, such as in the pulmonary anatomy, it may be beneficial to have the SDU 212 generate a different vacuum level, such as approximately −18 inHg in one situation. To reduce the vacuum level, the vacuum regulator 267 can be incorporated into the system to control and stabilize the vacuum supplied to the canister 218 and optionally accommodate for variations in elevation (and associated reduced air pressure at higher elevation) where the system is being operated. The vacuum regulator 267 can be a manually-adjustable unit that uses a spring force balanced against an internal diaphragm valve to compensate for fluctuations in downstream flow. The diaphragm has atmospheric pressure on one side and the regulated vacuum on the other side, resulting in the regulated vacuum level is compensated for changes in elevation (i.e., the canister vacuum, if set to −20 inHg at sea level, would still contain −20 inHg at 5280 feet elevation). More generally, the vacuum regulator 267 allows adjustment of a canister vacuum level, as measured by a canister vacuum sensor 219 that communicates with the controller 335, from the maximum attainable (described above) down to zero (atmospheric pressure).

The vacuum regulator 267 can be adjusted to a nominal −18 inHg setpoint during the manufacturing process, after which the setpoint can be mechanically locked, such as by a fastener, cable tie, etc., or locked using other techniques, to prevent inadvertent change or adjustment. The vacuum regulator 267 can be installed internally within the SDU 212, with the SDU case 284 preventing unauthorized access to the vacuum regulator 267 using tamper-evident seals or other security mechanisms or structures. Alternatively, the vacuum regulator 267 can be disposed externally to the SDU case 284 and can optionally remain unlocked. In still another configuration, the vacuum regulator 267, whether within or without the SDU case 284, can be electronically controlled by the controller 335, including associated hardware, firmware, software, etc. so that optimal vacuum levels for a particular aspiration catheter used with the system 200 or a particular patient anatomy can be automatically set, and optionally monitored by the controller 335 and other sensors disposed at any location within the system 200.

With continued reference to use of the system 200, a user connects a first connector 270 of the sterile suction tubing 216 to an aspiration luer 271 of the aspiration catheter 202 (similar to luer 115), and connects the second connector 272 of the sterile suction tubing 216 to port 259a in the lid 260 of the canister 218. Connector 236 is then coupled to the mating receptacle 237 in the SDU 212 for communication with the control 233 and/or the pressure sensor 230. For instance, the connector 236 can be snapped into mating receptacle 237 in the SDU 212 for communication with elements of the control 233 and/or for communication with the pressure sensor 230, either via cable 234, and/or additional cables or wires. Alternatively, the connector 236 may couple to the mating receptacle 237 by clipping, friction fitting, vacuum fitting, or other means.

After allowing saline to purge through the supply tube 276, cassette 278, and injection tube 279 of the pump set 210, the user connects the luer connector 280 of the pump set 210 to a luer 282 of the aspiration catheter 202 (similar to luer 113). The cassette 278 (similar to cassette 121) is then attached to a saddle 283 in the SDU 212. The saddle 283 is configured to reciprocate a piston to inject the saline from the IV bag 224 at high pressure, after the cassette 278 is snapped in place, keeping the internal contents (e.g., saline) sterile. Systems configured for performing this type of sterile injection of high-pressure saline are described in U.S. Pat. No. 9,883,877, issued Feb. 6, 2018, and entitled, “Systems and Methods for Removal of Blood and Thrombotic Material”, which is incorporated by reference in its entirety for all purposes. The SDU 212 is enclosed within a case 284 and a case lid 285. The controller 235 may reside on a circuit board 286 with associated processors, hardware, firmware, software, etc. Noise from a motor 287 controlling the saddle 283 and from the vacuum pump 266 is abated by internal foam sections 288, 289. The saddle 283 may be moved directly by the motor 287, or may be moved with pneumatics, using a cycled pressurization. An interface panel 290 provides one or more switches 297 and the display 238. Alternatively, the cassette 278 may couple to the saddle 283 by clipping, friction fitting, vacuum fitting, or other means.

The controller 235 operates the motor 287 to control movement of the saddle 283 and so move the piston 122 (FIG. 1) within the cassette 278 to pressurize fluid and deliver it to the aspiration catheter 202. The jet pressure from the opening 194 (FIG. 2) is proportional to the speed at which the motor 287 is driven and in one configuration the controller 235 can operate the motor 287 to operate in a range from about 280 rotations per minute (RPM) to about 340 RPM, resulting in jet pressures ranging from about 410 pounds per square inch (PSI) to about 707 PSI. Depending upon the particular anatomy within which the system 200 is used, the motor 287 can be operated at different speeds. For instance, for pulmonary anatomy, a desired jet pressure can be achieved by running the motor 287 at a reduced speed of 310 RPM. The controller 235 can be operated through the one or more switches 297 to vary a speed of the motor 287 based upon the particular patient anatomy, such as varying the speed of the motor 287 between about 310 RPM to about 340 RPM, from about 280 RPM to about 410 RPM, about 280 RPM, 290 RPM, 300 RPM, 310 RPM, 320 RPM, 330 RPM, 340 RPM, 350 RPM, 360 RPM, 370 RPM, 380 RPM, 390 RPM, 400 RPM, 410 RPM, or within a range between any of the proceeding. Additionally, or as an alternate to the switches, the controller 235 can adjust the motor speed using a proportional-integral-derivative (PID) speed control algorithm or feedback loop to monitor and correct a speed of the motor 287 for any changes in load conditions.

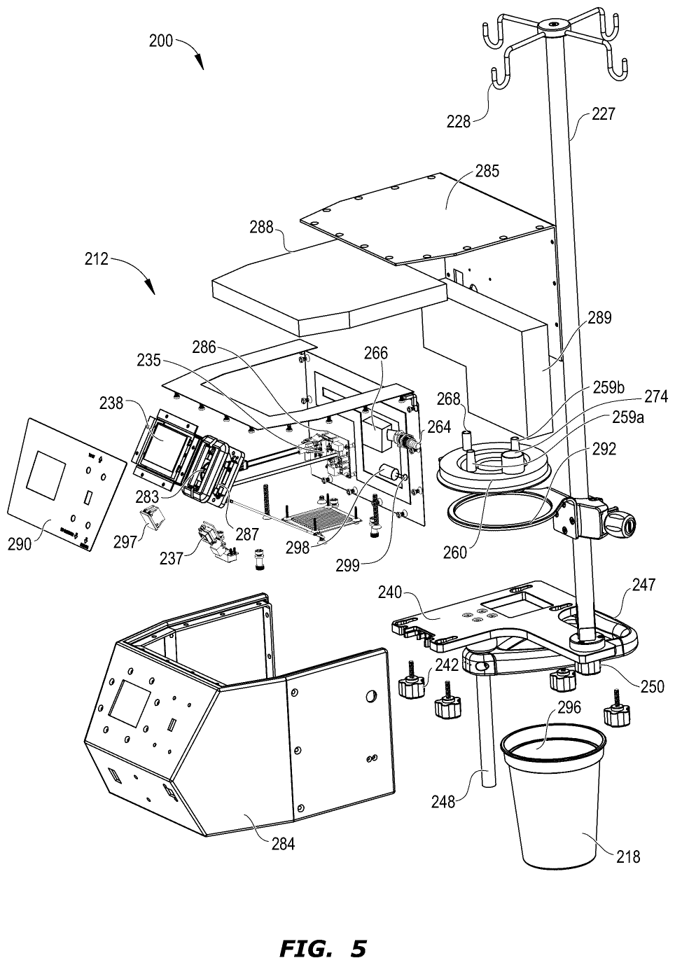

FIG. 5 illustrates aspects pertaining to the vacuum canister 218, in which aspirant (e.g., thrombus, blood, saline) that is evacuated from the patient through the aspiration lumen 106 is collected. The canister 218 may be held in a canister mount 292 carried by the IV pole 227, or alternatively carried by any other part of the system 200. A lid 260 is configured to cover a portion of the canister 218, such as in a snapping manner, to close an interior 296 of the canister 218. Alternatively, the lid 260 may couple to the canister 218 by screwing, clipping, friction fitting, or other means.

The lid 260 may comprise two or more ports, including the first port 259a and second port 259b for providing negative pressure/vacuum to the aspiration lumen 106. For example, the lid 260 may comprise two ports, three ports, four ports, or more than four ports. Sterile suction tubing 216 may be connected to the lid 260 of the vacuum canister 218 at a first port 259a for transmitting a negative pressure to the sterile suction tubing 216 and to the aspiration lumen 106 of the aspiration catheter 102. Non-sterile suction tubing 217 may be connected to the lid 260 of the vacuum canister 218 at a second port 259b for providing a negative pressure to the vacuum canister 218. A negative pressure may be provided to the non-sterile suction tubing 217 (and to sterile suction tubing 216 and the aspiration lumen 106 connected therewith) by a vacuum source (e.g., a vacuum pump or syringe). The system 200 may also comprise means for sealing the two or more ports of the lid 260 when not in use, such as one or more port caps. A filter may be placed over an entry to the second port 259b so as to prevent aspirant from traveling along the non-sterile suction tubing 217 from the vacuum canister 218 to the vacuum source.

The vacuum canister 218 preferably has a sufficient volumetric capacity for receiving all aspirant collected during the surgical procedure. Receptacles having a volumetric capacity of approximately 100 cubic inches, or receptacles having a diameter of approximately 5.0 inches and a height of approximately 7.0 inches, have been found to provide sufficient volumetric capacity.

Returning to FIG. 5, a solenoid 298 is carried internally in the SDU 212, and is configured to interface with the interior 296 of the canister 218, via the suction tubing 214, or via any additional tubing. The solenoid 298 is configured to vent the negative pressure inside the canister 218, by opening a valve 299 coupled to the solenoid (mechanically or electromagnetically) that opens the interior 296 of the canister 218 to ambient pressure. The venting allows any foaming of blood or fluid, such as any aspirated liquid, within the canister 218 to be reduced. Foaming can occur during a thrombolysis procedure due to cavitation, as air bubbles are formed. The solenoid 298 is then configured to close the valve 299, to allow negative pressure to again be built up within the interior 296 of the canister 218. The controller 235 is configured to automatically energize the solenoid 298, in order to allow for the degassing/defoaming. For example, the controller 235 may send a signal to energize the solenoid 298 based on the measurement of a targeted negative pressure and/or a targeted time of aspiration cycle. In other cases, the controller 235 can send a signal to energize the solenoid 298 every minute, every five minutes, every ten minutes, etc. Additionally, a user can operate the controller 235, and more generally the controller 174, of the system 200 through the interface panel 290 to initiate degassing/defoaming of the interior 296. The venting may also be able to remove air bubbles inside the other lumens of the catheter and tubing sets.

In some embodiments, the controller 235 can output or send a signal to energize the solenoid 298 to open the valve 299, in order to stop any aspiration, while still allowing the SDU 212 to deliver saline, medication, or saline combined with medication (e.g., thrombolytic drugs), so that the fluids can be delivered out of the open distal end 107 (instead of being aspirated through the aspiration lumen 106).

In addition to the above, the controller 235 more generally control the operation and functionality of the SDU 212 and the system 200 as a whole. An operation of the vacuum pump 266, such as operating speed, etc., can be controlled by the controller 235 (circuit board, processors, and associated or operatively connected hardware, firmware, etc.). While reference is made to noise from the motor 287 controlling the saddle 283 is abated by internal foam sections 288, 289, where a lower audible noise of the vacuum pump 266 might be more desirable for a user, the controller 235 can vary the operating speed of the vacuum pump 266 to reduce the audible noise of the system 200, and more particularly noise from the vacuum pump 266. For instance, the controller 235 can operate the vacuum pump 266 at a reduced speed, such as approximately 60% of maximum speed, during start-up and then reduce the speed to about 30% of maximum when a desired vacuum is achieved, such as −27.5 inHg. The controller 235, and associated printed circuit board and other hardware, firmware, etc., controls the vacuum pump speed at a fixed setpoint. A voltage divider circuit on the board produces a speed input signal to the vacuum pump 266 (such as a vacuum pump motor controller), which sets the pump speed based upon the speed input signal, such as to approximately 60% of maximum in this particular configuration. Other start-up speeds can be achieved with other speed input signals. For instance, the start-up speed can range from about 10% to about 100%, from about 20% to about 90%, from about 30% to about 80%, from about 40% to about 70%, from about 50% to about 60%, or within a range between any of the proceeding. Similarly, the operating speed of about 30% of maximum can also be varied in a similar manner so that the operating speed can range from 10% to about 100%, from about 20% to about 90%, from about 30% to about 80%, from about 40% to about 70%, from about 50% to about 60%, or within a range between any of the proceeding.

In still another configuration, control of the vacuum pump 266 can be achieved through the one or more switches 297 in combination with the controller 235. For instance, one of the switches 297 is a manually operated potentiometer that can vary the speed of the vacuum pump 266 from about 0% to about 100% of maximum speed. The speed setting from the potentiometer can be monitored to measure the feedback voltage, and optionally present speed and potentiometer information to the user through the display 238.

Additionally, the controller 235 controls the information presented on the display 238 or through the SDU 212, such as alarms, warnings, pressure and flow information, or any other information, warnings, etc. related to the operation of the system 200. For instance, the controller 235 can include hardware, firmware, etc. that provides through the display, etc. notification of various alarms or other information, such as (i) a “No Suction” alarm notifying a user of vacuum leaks in the system, (ii) a “Terminal Vacuum Fault” alarm indicating a vacuum level that is too high or different from a predetermined threshold which occurs when the vacuum regulator 267 has failed, such as when the canister vacuum is lower than −20 inHg, lowerthan −18 inHg or some other predetermined threshold, (iii) “Terminal Motor Fault” alarm indicating a problem with operation of the cassette 278, and (iv) a splash screen, which displays for a few seconds upon power-up of the system 200, providing version information or other relevant information related to any of the hardware, firmware, etc. of the controller 235 or another component of the SDU 212.

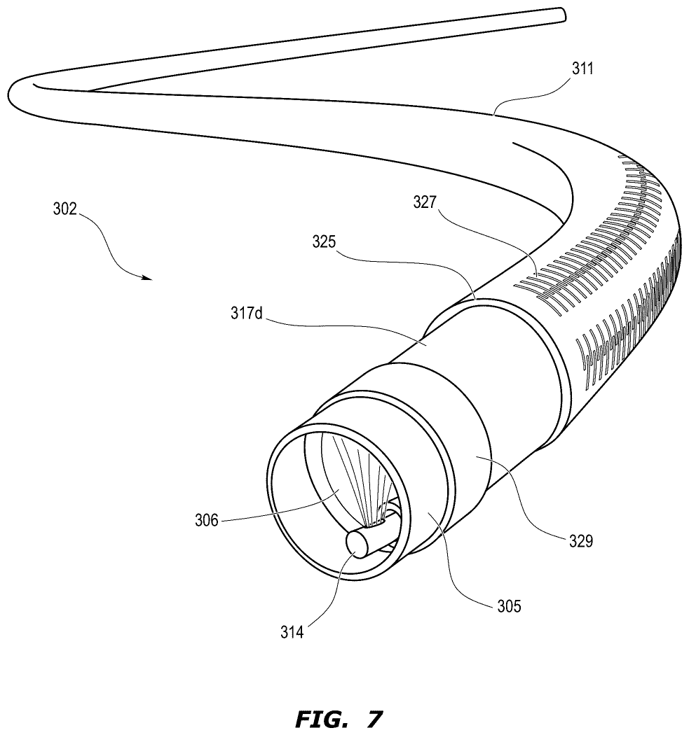

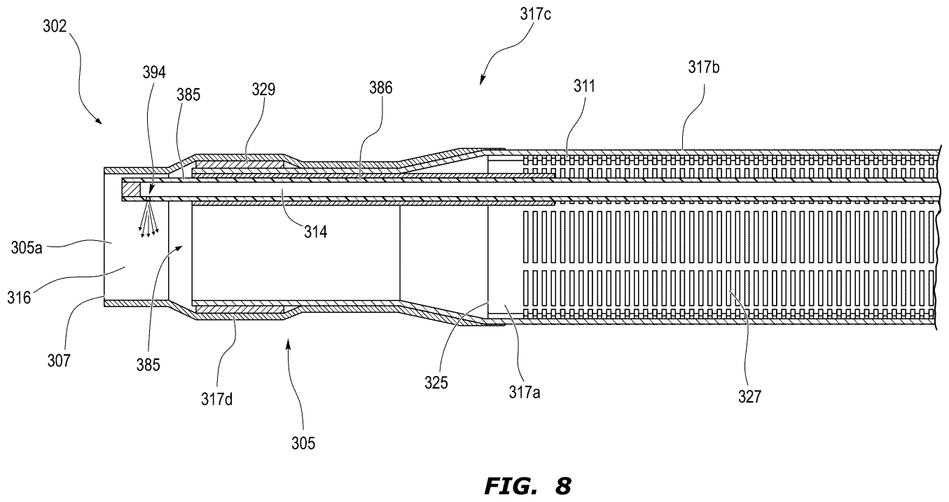

Turning to FIGS. 7-9 illustrated is another configuration of an aspiration catheter 302 that can be used with the systems 100 or 200 for aspirating thrombus. As such, the discussions related to the aspiration catheter 102 and the aspiration catheter 202 are also applicable to the aspiration catheter 302 illustrated in FIGS. 7-9.

As shown in more detail in FIGS. 7-9, aspiration catheter 302 includes an aspiration lumen 306 formed by a shaft 311, such as a hypotube, jacketed by a polymer jacket or a polymer jacket is laminated on the hypotube. For instance, a shaft body 317a is illustrated being jacketed by a jacket 317b. A distal end 305 of the aspiration catheter 302 includes a multilayer structure. A portion 317c of the jacket 317b extends distally of a shaft distal end 325 of the shaft 311 to form part of the distal end 305. An outer layer or outer jacket 317d overlaps the jacket 317b, extends towards and overlaps a shaft distal end 325, and forms the aspiration catheter distal end 305 or the distal most end of the aspiration catheter with the distal opening 307. The outer jacket 317d protects a distal portion 385 of a supply tube 386 containing a supply lumen 314. While reference is made to a multilayer structure, it will be understood that one or more layers can be omitted or combined together. Additionally, one or more of the layers or shaft body can include braided or other members to increase strength and/or flexibility. Alternatively, the shaft and associated layers can be formed by extruding the shaft or using other structure to form the shaft.

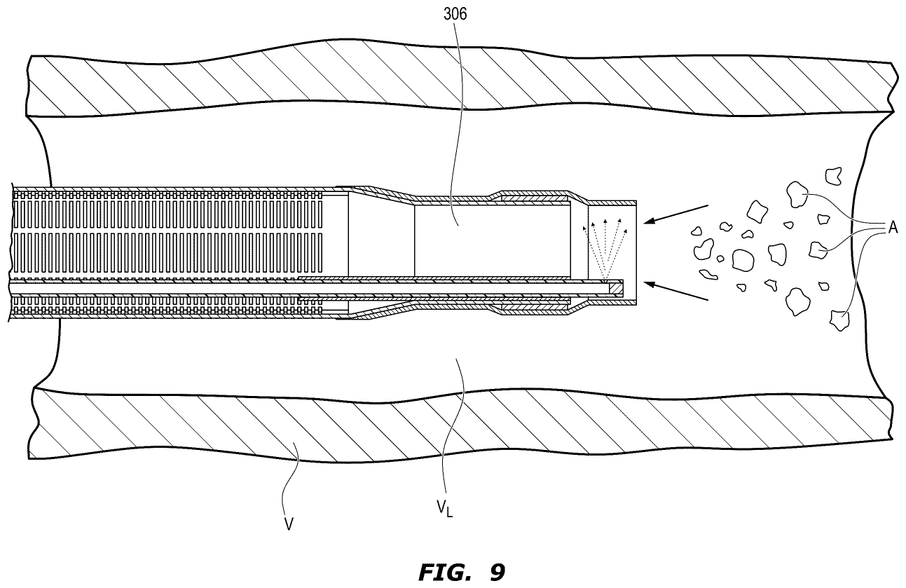

The supply lumen 314 may be configured to provide a high pressure fluid injection, such as saline, within the aspiration lumen 306 for macerating a thrombus as it is aspirated, such as illustrated in FIG. 9 where the aspiration catheter 302 is disposed within a vessel lumen VL of a vessel V and aspirant A is being drawn into the aspiration lumen 306. The saline injection may occur through orifice 394 near the distal end of the supply lumen 314 if the opening of the supply lumen is plugged with a plug similar to plug 192 (FIG. 2). Aspiration catheter 302 may also include a radiopaque (RO) ring 329 at or near the distal end 305 of aspiration catheter 302 for identifying the location of aspiration. The radiopaque ring 329 optionally encircles the aspiration catheter 302. In the illustrated configuration, the RO ring 329 is disposed between the jacket 317b and outer jacket 317d. The RO ring 329 can be formed of or comprise any suitable radiopaque material, such as tantalum, tungsten, platinum/iridium, gold, silver, and combinations or modifications thereof.

The shaft 311 can include one or more openings 327 to increase a flexibility of shaft 311 to aid with advancement of the aspiration catheter 302 through the tortuous anatomy of a patient. While reference is made to a “hypotube,” it will be understood that other tubular structures can be used for the shaft 311. Additionally, the shaft 311 can be formed from polymers, metals, alloys, braided structures, coiled structures, and combinations or modifications thereof. Furthermore, the jacket 317b and outer jacket 317d can be formed of a variety of polymers and copolymers, plastics, PEBAX, HYTREL, rubber, thermoplastic elastomer, other elastomer and combinations or modifications thereof.

It should be appreciated that the supply lumen may include any number of orifices, e.g., from 1 to 10, from 2 to 8, or from 3 to 6, such as 2, 3, 4, 5, 6, 7, 8, 9, or 10. Where multiple orifices are provided such orifices may be provided across multiple supply lumens, for example, equally or unequally spaced about the aspiration catheter. In addition, such orifices may be positioned different distances from the distal opening of the aspiration catheter (e.g., some orifices recessed a greater distance from the distal opening than others.

Furthermore, while the Figures may show the one or more orifices formed as a radial opening in the wall of the supply tube, with the wall(s) that forms the orifice between inner and outer surfaces of the supply tube being generally perpendicular to the tangential axis of the wall, this is not required. For example, alternatively, in some embodiments, an orifice for forming a fluid jet for an aspiration catheter of a system may be formed as an angled opening, where the orifice wall(s) that forms the orifice (between inner and outer surfaces of a material) has an acute or obtuse angle relative to the tangential axis of the material in which the orifice is formed (e.g., material of a supply tube, or a component of the supply tube at a distal end thereof, in which the orifice(s) are formed). For example, the angle between the orifice wall(s) and the tangential axis of the material on which the orifice is formed can be within a range of 20° to 70°, or 30° to 60°, or 40° to 50° (or within their complementary ranges).

The orifice(s) of an aspiration catheter of a system for providing fluid jets as described herein may be implemented with various sizes or shapes in different embodiments. In one example, an orifice may comprise a circular hole with a diameter between 0.03 mm (0.001 inches) and 0.15 mm (0.006 inches), or between about 0.0508 mm (0.002 inches) and about 0.1016 mm (0.004 inches), or about 0.0787 mm (0.0031 inches). The diameter of the supply lumen 114 may be between about 0.3048 mm (0.012 inches) and about 0.4826 mm (0.019 inches), or between about 0.3556 mm (0.014 inches and about 0.4318 mm (0.017 inches), or within any range having end points within any one or combination of the foregoing ranges. As another example, an orifice may comprise a rectangular hole with each side thereof having a length between 0.02 mm (0.0008 inches) and 0.20 mm (0.008 inches). In some implementations, the total cross-sectional area of all jet-forming orifices of an aspiration catheter of a system is between 0.002 mm² and 0.02 mm², or between 0.003 mm² and 0.015 mm², or between 0.005 mm² and 0.01 mm². In some embodiments, pressure of the pressurized fluid may be between 400 psi (2.6 MPa) and 3,000 psi (20.7 MPa) 400 psi (2.6 MPa) and 2,000 psi (13.8 MPa), or between 500 psi (3.4 MPa) and 2,000 psi (13.8 MPa), or between 600 psi (4.1 MPa) and 1,750 psi (12.1 MPa). Such pressures may be as measured at a proximal location, e.g., where the pressurized fluid enters the supply lumen, or at a distal location, e.g., where it exits the orifice(s).

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

As used herein, the term “between” includes referenced endpoints within any recited range. For example, the phrasing “between 0.010 inches and 50 inches” includes 0.010 inches and 50 inches.

Following are some further example embodiments of the invention. These are presented only by way of example and are not intended to limit the scope of the invention in any way. Further, any example embodiment can be combined with one or more of the example embodiments.

Embodiment 1. A thrombectomy system, comprising:
an aspiration catheter having an aspiration lumen and a supply lumen, the aspiration catheter having a proximal end and a distal end; and
a drive unit fluidly connected to the aspiration catheter and configured to provide pressurized fluid and vacuum through the aspiration catheter,
wherein, the aspiration catheter comprises a distal opening and wherein the supply lumen has at least one orifice, wherein the at least one distal orifice of the supply lumen is proximal of the distal opening of the aspiration catheter, so as to be proximally disposed relative to the distal opening, between about 0.035 inches and about 0.060 inches.

Embodiment 2. The system of embodiment 1, wherein the at least one distal orifice is proximal of the distal opening between about 0.040 inches and about 0.055 inches.

Embodiment 3. The system of any of embodiments 1-2, wherein the at least one orifice is configured to form a spray pattern of the pressurized fluid delivered along the supply lumen.

Embodiment 4. The system of any of embodiments 1-3, wherein the drive unit comprises a vacuum regulator.

Embodiment 5. A method of removing a thrombus, comprising the steps of:
providing the system of any of embodiments 1-4;
inserting the aspiration catheter percutaneously into a body vessel lumen;
advancing the aspiration catheter to a region of interest; and
operating the drive unit to provide pressurized fluid and aspiration through the aspiration catheter to draw the thrombus into the aspiration lumen.

Embodiment 6. A thrombectomy system, comprising
an aspiration catheter having an aspiration lumen and a supply lumen, the aspiration catheter having a proximal end and a distal end; and
a drive unit fluidly connected to the aspiration catheter and configured to provide pressurized fluid and vacuum through the aspiration catheter,
wherein, the aspiration catheter comprises a distal opening and wherein the supply lumen has at least one orifice, wherein the at least one distal orifice of the supply lumen is proximal of the distal opening of the aspiration catheter, so as to be proximally disposed relative to the distal opening between about 0.010 inches and about 50 inches.

Embodiment 7. The system of embodiment 6, wherein the at least one orifice is proximal of the distal opening between about 0.010 inches to about 0.200 inches.

Embodiment 8. The system of any of embodiments 6-7, wherein the aspiration catheter has a size ranging from about 3 Fr to about 8 Fr.

Embodiment 9. The system of any of embodiments 6-8, wherein the at least one distal orifice is proximal of the distal opening between about 0.010 inches to about 2 inches.

Embodiment 10. The system of any of embodiments 6-9, wherein the aspiration catheter has a size ranging from about 9 Fr to about 11 Fr.

Embodiment 11. The system of any of embodiments 6-10, wherein the at least one distal orifice is proximal of the distal opening between 0.010 inches to about 40 inches.

Embodiment 12. The system of any of embodiments 6-11, wherein the aspiration catheter has a size ranging from about 12 Fr to about 16 Fr.

Embodiment 13. The system of any of embodiments 6-12, wherein the at least one distal orifice is proximal of the distal opening between 0.010 inches to about 50 inches.

Embodiment 14. The system of any of embodiments 6-13, wherein the aspiration catheter has a size ranging from about 17 Fr to about 26 Fr.

Embodiment 15. The system of any of embodiments 6-14, wherein the drive unit comprises a vacuum regulator.

Embodiment 16. The system of any of embodiments 6-15, wherein the vacuum regulator accommodates for variations in elevation within a location where the system is being operated.

Embodiment 17. The system of any of embodiments 6-16, further comprising a pump operatively coupled to the supply lumen, the pump having an adjustable or variable speed.

Embodiment 18. The system of any of embodiments 6-17, wherein a controller of the drive unit controls and monitors a speed of the pump to maintain the speed within a predetermined range.

Embodiment 19. The system of any of embodiments 6-18, wherein a controller of the drive unit controls and monitors a speed of the pump.

Embodiment 20. The system of any of embodiments 6-19, further comprising a pump operatively coupled to a canister in fluid communication with the aspiration lumen.

Embodiment 21. The system of any of embodiments 6-20, wherein a controller of the drive unit controls the pump to operate between about 30% of maximum speed and about 100% maximum speed.

Embodiment 22. The system of any of embodiments 6-21, wherein the controller of the drive unit controls the pump to operate at about 60% of maximum speed during start-up of the drive unit.

Embodiment 23. The system of any of embodiments 6-22, wherein the controller comprises a potentiometer.

Embodiment 24. The system of any of embodiments 6-23, further comprising a controller operatively coupled to a display, wherein the controller is configured to provide at least one of alarms, warnings, pressure, or flow information, or other information via the display.

Embodiment 25. A method of removing a thrombus, comprising the steps of:
providing the system of any of embodiments 6-24;
inserting the aspiration catheter percutaneously into a body vessel lumen;
advancing the aspiration catheter to a region of interest; and
operating the drive unit to provide pressurized fluid and aspiration through the aspiration catheter to draw the thrombus into the aspiration lumen.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A thrombectomy system, comprising
an aspiration catheter having an aspiration lumen and a supply lumen, the aspiration catheter having a proximal end and a distal end; and
a drive unit fluidly connected to the aspiration catheter and configured to provide pressurized fluid and vacuum through the aspiration catheter,
wherein, the aspiration catheter comprises a distal opening and wherein the supply lumen has at least one orifice, wherein the at least one distal orifice of the supply lumen is proximal of the distal opening of the aspiration catheter, so as to be proximally disposed relative to the distal opening, between about 0.035 inches and about 0.060 inches.

2. The system of claim 1, wherein the at least one distal orifice is proximal of the distal opening between about 0.040 inches and about 0.055 inches.

3. The system of claim 1, wherein the at least one orifice is configured to form a spray pattern of the pressurized fluid delivered along the supply lumen.

4. The system of claim 1, wherein the drive unit comprises a vacuum regulator.

5. A method of removing a thrombus, comprising the steps of:
providing the system of claim 1;
inserting the aspiration catheter percutaneously into a body vessel lumen;
advancing the aspiration catheter to a region of interest; and
operating the drive unit to provide pressurized fluid and aspiration through the aspiration catheter to draw the thrombus into the aspiration lumen.

6. A thrombectomy system, comprising
an aspiration catheter having an aspiration lumen and a supply lumen, the aspiration catheter having a proximal end and a distal end; and
a drive unit fluidly connected to the aspiration catheter and configured to provide pressurized fluid and vacuum through the aspiration catheter,
wherein, the aspiration catheter comprises a distal opening and wherein the supply lumen has at least one orifice, wherein the at least one distal orifice of the supply lumen is proximal of the distal opening of the aspiration catheter, so as to be proximally disposed relative to the distal opening between about 0.010 inches and about 50 inches.

7. The system of claim 6, wherein the at least one orifice is proximal of the distal opening between about 0.010 inches to about 0.200 inches.

8. The system of claim 7, wherein the aspiration catheter has a size ranging from about 3 Fr to about 8 Fr.

9. The system of claim 6, wherein the at least one distal orifice is proximal of the distal opening between about 0.010 inches to about 2 inches.

10. The system of claim 7, wherein the aspiration catheter has a size ranging from about 9 Fr to about 11 Fr.

11. The system of claim 6, wherein the at least one distal orifice is proximal of the distal opening between 0.010 inches to about 40 inches.

12. The system of claim 11, wherein the aspiration catheter has a size ranging from about 12 Fr to about 16 Fr.

13. The system of claim 6, wherein the at least one distal orifice is proximal of the distal opening between 0.010 inches to about 50 inches.

14. The system of claim 13, wherein the aspiration catheter has a size ranging from about 17 Fr to about 26 Fr.

15. The system of claim 6, wherein the drive unit comprises a vacuum regulator.

16. The system of claim 15, wherein the vacuum regulator accommodates for variations in elevation within a location where the system is being operated.

17. The system of claim 6, further comprising a pump operatively coupled to the supply lumen, the pump having an adjustable or variable speed.

18. The system of claim 17, wherein a controller of the drive unit controls and monitors a speed of the pump to maintain the speed within a predetermined range.

19. The system of claim 17, wherein a controller of the drive unit controls and monitors a speed of the pump.

20. The system of claim 6, further comprising a pump operatively coupled to a canister in fluid communication with the aspiration lumen.

21. The system of claim 20, wherein a controller of the drive unit controls the pump to operate between about 30% of maximum speed and about 100% maximum speed.

22. The system of claim 21, wherein the controller of the drive unit controls the pump to operate at about 60% of maximum speed during start-up of the drive unit.

23. The system of claim 19, wherein the controller comprises a potentiometer.

24. The system of claim 7, further comprising a controller operatively coupled to a display, wherein the controller is configured to provide at least one of alarms, warnings, pressure, or flow information, or other information via the display.

25. A method of removing a thrombus, comprising the steps of:
providing the system of claim 6;
inserting the aspiration catheter percutaneously into a body vessel lumen;
advancing the aspiration catheter to a region of interest; and
operating the drive unit to provide pressurized fluid and aspiration through the aspiration catheter to draw the thrombus into the aspiration lumen.
